(19) United States
(12) Patent Application Publication
(10) Pub. No.: US 2026/0207369 A1
(43) Pub. Date: Jul. 23, 2026

(54) Nasal Breathing Device

(71) Applicant: Michael Kevin McKiney, Pennsauken Township, NJ (US)

(72) Inventor: Michael Kevin McKiney, Pennsauken Township, NJ (US)

(21) Appl. No.: 19/401,474

(22) Filed: Nov. 26, 2025

Related U.S. Application Data
(60) Provisional application No. 63/747,498, filed on Jan. 21, 2025.

(51) Int. Cl. A61F 5/08

(52) U.S. Cl. CPC A61F 5/08

(57) ABSTRACT

The subject matter disclosed and claimed herein, in one embodiment thereof, comprises an improved breather device. The device is an insertable device designed to open the nasal passages for improved breathing and air flow. The device has two vented nasal inserts that are round in shape to be inserted into the nostrils in order to expand them. The nasal inserts are linked together by a hook which dangles at the base of the nose. The hook enables easy insertion, adjustment, and removal of the device in and out of the nostrils as needed. The device comprises multiple sizes for different size noses and nostrils. Thus, once inserted into the nose, the device opens the nasal passages and enables more air flow into the lungs as the user breathes.

### CROSS-REFERENCE TO RELATED APPLICATION

The present application claims priority to, and the benefit of, U.S. Provisional Application No. 63/747,498, which was filed on Jan. 21, 2025, and is incorporated herein by reference in its entirety.

### FIELD OF THE INVENTION

The present invention relates generally to the field of breathing aid devices. More specifically, the present invention relates to a breathing aid to hold the nares open, promoting better airflow into the lungs. Accordingly, the present disclosure makes specific reference thereto. Nonetheless, it is to be appreciated that aspects of the present invention are also equally applicable to other like applications, devices, and methods of manufacture.

### BACKGROUND

By way of background, this invention relates to improvements in breathing aid devices. Generally, individuals that are exercising or participating in sporting events may experience lack of airflow, especially through the nostrils. Lack of airflow while training can slow down blood flow and cause athletes to get tired quickly. Allergies can cause nasal passages to close because of mucus, and constantly blowing one's nose can be frustrating and uncomfortable. While sleeping, individuals may experience closed nasal passages, thereby opening their mouth and potentially snoring.

Accordingly, there is a demand for an improved breathing aid device that holds the nares open and encourages more airflow into the lungs. More particularly, there is a demand for a correct breather device that aids in breathing while sleeping or exercising.

Therefore, there exists a long-felt need in the art for a correct breather device that provides users with a nasal insert device that helps the wearer breathe optimally while exercising, sleeping, and more. There is also a long-felt need in the art for a correct breather device that utilizes a hook mechanism to link together two round sections with holes, enabling users to conveniently insert the device into the nostrils. Further, there is a long-felt need in the art for a correct breather device that maintains easy adjustment of the device to ensure comfort when it is being worn. Finally, there is a long-felt need in the art for a correct breather device that widens the nasal passages to provide greater air intake into the lungs.

The subject matter disclosed and claimed herein, in one embodiment thereof, comprises a correct breather device. The device is an insertable device designed to open the nasal passages for improved breathing and air flow. The device has two vented nasal inserts that are round in shape to be inserted into the nostrils to expand them. The nasal inserts are linked together by a hook which dangles at the base of the nose. The hook enables easy insertion, adjustment, and removal of the device in and out of the nostrils as needed. The device comprises multiple sizes for different size noses and nostrils. Thus, once inserted into the nose, the device opens the nasal passages and enables more air flow into the lungs as the user breathes. Accordingly, the device can be used in various situations for exercise, sleeping, and much more.

In this manner, the breather device of the present invention accomplishes all of the forgoing objectives and provides users with a device that promotes greater airflow into the lungs during various activities.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a correct breather device. The device is an insertable device designed to open the nasal passages and nares for improved breathing and air flow. The device has two vented nasal inserts that are round in shape to be inserted into the nostrils to expand them. The nasal inserts are linked together by a hook which dangles at the base of the nose. The hook enables easy insertion, adjustment, and removal of the device in and out of the nostrils as needed. The device comprises multiple sizes for different size noses and nostrils. Thus, once inserted into the nose, the device opens the nasal passages and enables more air flow into the lungs as the user breathes.

In one embodiment, the breather device comprises two nostril inserts. The nostril inserts are typically configured in a circular shape but can be configured in any suitable shape as is known in the art. Typically, both of the nostril inserts are vented and comprise grilles for structural stability.

In one embodiment, the nostril inserts are connected by a hook that, in use, dangles at the base of the nose. The hook is typically configured in a U-shape and is flexible so that the device can be expanded or contracted (i.e., a distance between the nostril inserts) to comfortably fit different shapes and sizes of noses. Typically, the hook comprises a thicker grip at the bottom for easy grasping while adjusting.

In one embodiment, the breather device in accordance with the present invention can be produced in various colors, designs, patterns, etc., and feature logos, emblems and or designs, such as a distributor requests and/or a user desires.

In one embodiment, the breather device is manufactured of a high-density polyethylene (HDPE), or any other suitable materials as is known in the art. Any number of different types of materials can be used to make the breather device including but not limited to plastics, nylon, etc.

In use, the user inserts the two nostril inserts into their nostrils. The fit of the device can be adjusted by the user by moving the grip and/or hook that connects the nostril inserts. The user can then utilize the device to encourage efficient breathing while exercising, sleeping, or other activities.

It will also be appreciated that there are a number of additional add-on features that can be incorporated into the device and moreover, the breather device can take many different forms as is known in the art.

In yet another embodiment, the breather device comprises a plurality of indicia.

In yet another embodiment, a method of conveniently utilizing the breather device to encourage efficient breathing is disclosed. The method includes the steps of providing a correct breather device comprising two nostril inserts connected by a hook. The method also comprises inserting the nostril inserts into the nostrils. Further, the method comprises adjusting the device by moving the hook and attached grip. Finally, the method comprises utilizing the device to encourage efficient breathing while exercising, sleeping, or other activities.

Numerous benefits and advantages of this invention will become apparent to those skilled in the art to which it pertains, upon reading and understanding the following detailed specification.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters

refer to similar parts throughout the different views, and in which:

FIG. 1 illustrates a perspective view of one embodiment of the breather device of the present invention in accordance with the disclosed architecture;

FIG. 2 illustrates a perspective view of one embodiment of the breather device of the present invention showing the device being expanded in accordance with the disclosed architecture;

FIG. 3 illustrates a perspective view of one embodiment of the breather device of the present invention showing the device being contracted (i.e., a distance between the nostril inserts) in accordance with the disclosed architecture;

FIG. 4 illustrates a perspective view of one embodiment of the breather device of the present invention showing the device inserted into the nostrils of a user in accordance with the disclosed architecture;

FIG. 5 illustrates a perspective view of one embodiment of the breather device of the present invention showing how widened nasal passages enable for improved airflow in accordance with the disclosed architecture; and

FIG. 6 illustrates a flowchart showing the method of conveniently utilizing the breather device to encourage efficient breathing in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention and do not limit the scope of the invention. Additionally, an illustrated embodiment need not have all the aspects or advantages shown. Thus, in other embodiments, any of the features described herein from different embodiments may be combined.

As noted above, there is a long-felt need in the art for a correct breather device that provides users with a nasal insert device that helps the wearer breathe optimally while exercising, sleeping, and more. There is also a long-felt need in the art for a correct breather device that utilizes a hook mechanism to link together two round sections with holes, enabling users to conveniently insert the device into the nostrils. Further, there is a long-felt need in the art for a correct breather device that maintains easy adjustment of the device to ensure comfort when it is being worn. Finally, there is a long-felt need in the art for a correct breather device that widens the nasal passages to provide greater air intake into the lungs.

The present invention, in one exemplary embodiment, is a novel correct breather device. The device is an insertable device designed to open the nasal passages for improved breathing and air flow. The device has two vented nasal inserts that are round in shape to be inserted into the nostrils to expand them. The nasal inserts are linked together by a hook which dangles at the base of the nose. The hook enables easy insertion, adjustment, and removal of the device in and out of the nostrils as needed. The device comprises multiple sizes for different size noses and nostrils. Thus, once inserted into the nose, the device opens the nasal passages and enables more air flow into the lungs as the user breathes. The present invention also includes a novel method of conveniently utilizing the breather device to encourage efficient breathing. The method includes the steps of providing a correct breather device comprising two nostril inserts connected by a hook. The method also comprises inserting the nostril inserts into the nostrils. Further, the method comprises adjusting the device by moving the hook and attached grip. Finally, the method comprises utilizing the device to encourage efficient breathing while exercising, sleeping, or other activities.

Referring initially to the drawings, FIG. 1 illustrates a perspective view of one embodiment of the breather device or breathing aid 100 of the present invention. In the present embodiment, the breather device or breathing aid 100 is an improved breather device or breathing aid 100 that provides a user 200 with a breathing aid to improve airflow while exercising, sleeping, or other activities Specifically, the breather device or breathing aid 100 comprises two nostril inserts 110 connected by a hook 120. Thus, once inserted into the nose, the device 100 opens the nasal passages and enables more airflow into the lungs as the user 200 breathes.

In the present embodiment, the breather device or breathing aid 100 comprises two nostril inserts 110. The nostril inserts 110 are typically configured in a circular shape but can be configured in any suitable shape as is known in the art. Typically, both of the nostril inserts 100 are vented and comprise grilles 112 for structural stability.

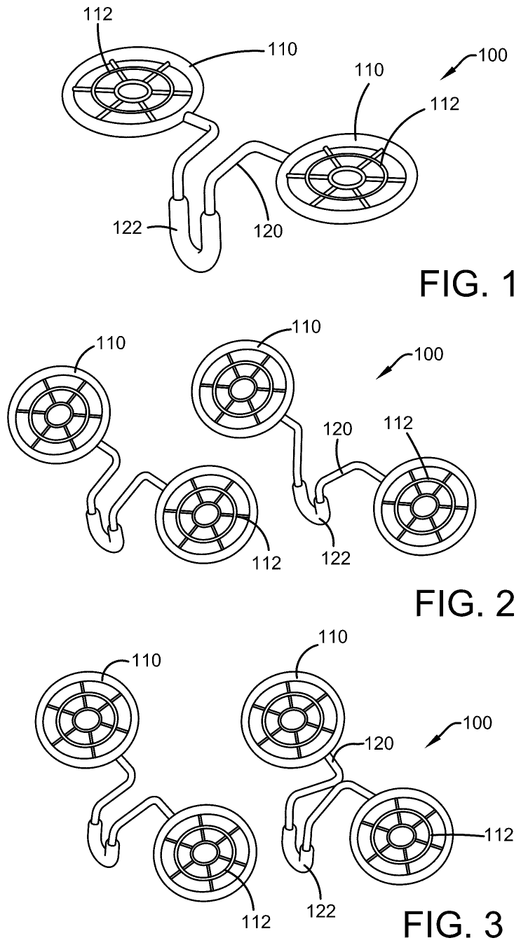

As shown in FIGS. 2 and 3, the nostril inserts 110 are connected by a hook 120 that, in use, dangles at the base of the nose. The hook 120 is typically configured in a U-shape and is flexible so that the device 100 can be expanded or contracted (i.e., a distance between the nostril inserts) to comfortably fit different shapes and sizes of noses. Typically, the hook 120 comprises a thicker grip portion 122 at the bottom, or mid-section, for easy grasping while adjusting.

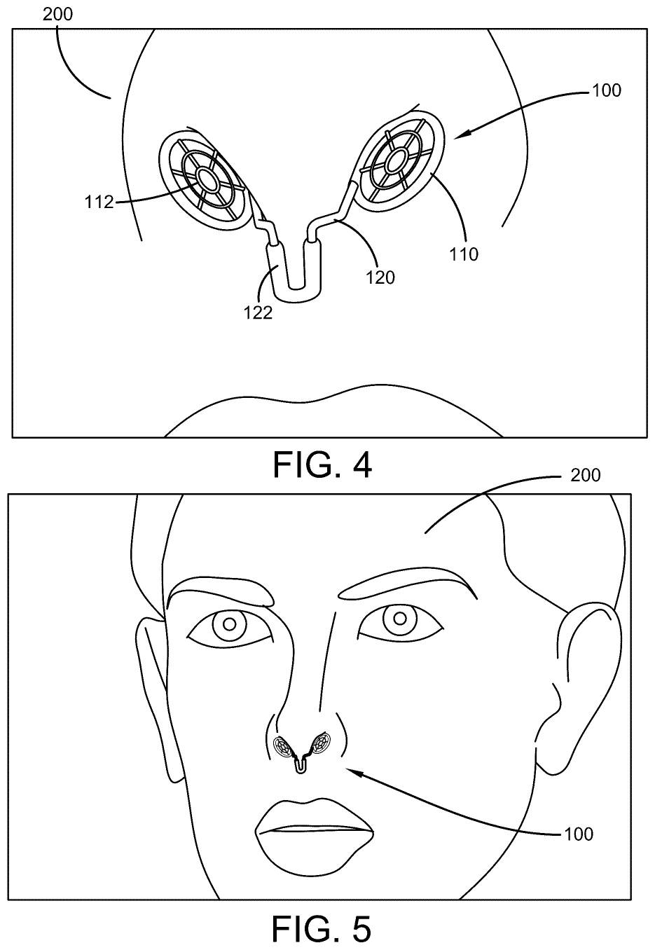

As shown in FIGS. 4 and 5, in use, the user 200 inserts the two nostril inserts 110 into their nostrils. The fit of the device 100 can be adjusted by the user 200 by moving the grip 122 and/or hook 120 that connects the nostril inserts 110 to adjust the distance between the two nostril inserts. The user 200 can then utilize the device 100 to encourage efficient breathing while exercising, sleeping, or other activities. Thus, once inserted into the nose, the device 100 opens the nasal passages and enables more air flow into the lungs as the user 200 breathes.

It is also to be appreciated that the breather device or breathing aid 100 in accordance with the present invention can be produced in various colors, designs, patterns, etc., and feature logos, emblems and or designs, such as a distributor requests and/or a user 200 desires.

In one embodiment, the breather device or breathing aid 100 is manufactured of a high-density polyethylene (HDPE), or any other suitable materials as is known in the art. Any number of different types of materials can be used to make the breather device or breathing aid 100 including but not limited to nylon, heat-sealable plastic or polymers, such as polypropylene or acrylonitrile-butadiene-styrene (ABS), or any other suitable material as is known in the art, such as but not limited to, acrylic, polycarbonate, polyethylene, polyethylene terephthalate, polyvinyl chloride, polystyrene, etc. Generally, the breather device or breathing aid 100 is also manufactured from a material that is water resistant and waterproof.

In yet another embodiment, the breather device or breathing aid 100 comprises a plurality of indicia 400. The hook 120 of the device 100 may include advertising, a trademark, or other letters, designs, or characters, printed, painted, stamped, or integrated into the hook 120, or any other indicia 400 as is known in the art. Specifically, any suitable indicia 400 as is known in the art can be included, such as but not limited to, patterns, logos, emblems, images, symbols, designs, letters, words, characters, animals, advertisements, brands, etc., that may or may not be nose, breathing, or brand related.

It will also be appreciated that there are a number of additional add-on features that can be incorporated into the device 100 and moreover, the breather device or breathing aid 100 can take many different forms as is known in the art.

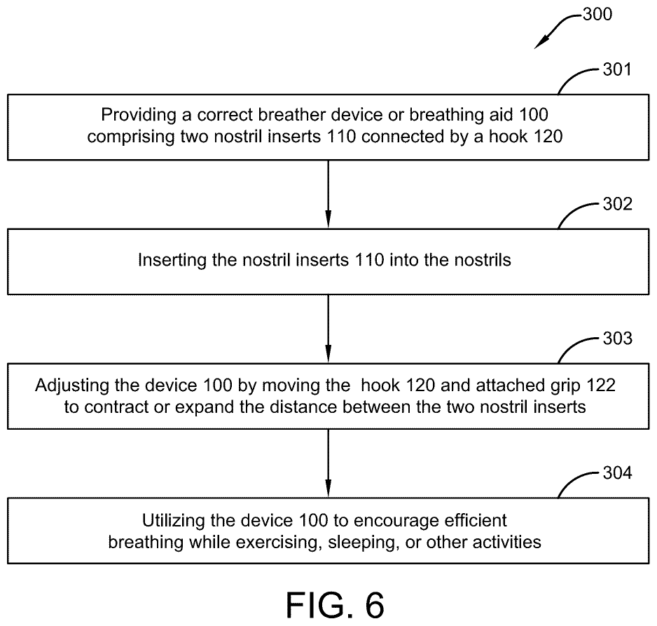

FIG. 6 illustrates a flowchart of the method 300 of conveniently utilizing the breather device or breathing aid 100 to encourage efficient breathing. The method 300 includes the steps of at 301, providing a correct breather device or breathing aid 100 comprising two nostril inserts 110 connected by a hook 120. The method 300 also comprises at 302, inserting the nostril inserts 110 into the nostrils. Further, the method 300 comprises at 303, adjusting the device 100 by moving the hook 120 and attached grip 122 to contract or expand the distance between the two nostril inserts. Finally, the method comprises at 304, utilizing the device 100 to encourage efficient breathing while exercising, sleeping, or other activities.

Certain terms are used throughout the following description and claims to refer to particular features or components. As one skilled in the art will appreciate, different users may refer to the same feature or component by different names. This document does not intend to distinguish between components or features that differ in name but not structure or function. As used herein “correct breather device”, “breathing aid device”, “breathing device”, and “device” are interchangeable and refer to the breather device or breathing aid 100 of the present invention.

Notwithstanding the forgoing, the breather device or breathing aid 100 of the present invention can be of any suitable size and configuration as is known in the art without affecting the overall concept of the invention, provided that it accomplishes the above stated objectives. One of ordinary skill in the art will appreciate that the breather device or breathing aid 100 as shown in FIGS. 1-5 is for illustrative purposes only, and that many other sizes and shapes of the breather device or breathing aid 100 are well within the scope of the present disclosure. Although the dimensions of the breather device or breathing aid 100 are important design parameters for user convenience, the breather device or breathing aid 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A breather device comprising:
a breathing aid having a pair of nostril inserts and a connecting hook;
wherein said connecting hook connects said pair of nostril inserts;
wherein one of said pair of nostril inserts insertable into a nostril; and
further wherein another one of said pair of nostril inserts insertable into another nostril for opening nasal passages of a wearer.

2. The breather device of claim 1, wherein each one of said pair of nostril inserts having a circular shape.

3. The breather device of claim 2, wherein each one of said pair of nostril inserts are vented.

4. The breather device of claim 3, wherein each one of said pair of nostril inserts comprise a grill structure.

5. The breather device of claim 4, wherein said connecting hook is flexible for expanding and contracting a distance between said pair of nostrils of said breathing aid.

6. The breather device of claim 5, wherein said connecting hook having a U-shape.

7. The breather device of claim 6, wherein said connecting hook having a grip at a mid-section of said connecting hook for grasping by the wearer.

8. The breather device of claim 7, wherein said mid-section of said connecting hook is thicker than end portions of said connecting hook.

9. A breather device comprising:
a breathing aid having a pair of nostril inserts and a connecting hook;
wherein said connecting hook connects said pair of nostril inserts;
wherein one of said pair of nostril inserts insertable into a nostril;
wherein another one of said pair of nostril inserts insertable into another nostril for opening nasal passages of a wearer;
wherein each one of said pair of nostril inserts are vented; and
further wherein each one of said pair of nostril inserts comprise a grill structure.

10. The breather device of claim 9, wherein each one of said pair of nostril inserts having a circular shape.

11. The breather device of claim 10, wherein said connecting hook is flexible for expanding and contracting a distance between said pair of nostrils of said breathing aid.

12. The breather device of claim 11, wherein said connecting hook having a U-shape.

13. The breather device of claim 12, wherein said connecting hook having a grip at a mid-section of said connecting hook for grasping by the wearer.

14. The breather device of claim 13, wherein said mid-section of said connecting hook is thicker than end portions of said connecting hook.

15. The breathing device of claim 14, wherein said breathing comprising a material selected from the group consisting of a high-density polyethylene (HDPE), a nylon, a heat-sealable polymer, a polypropylene and an acrylonitrile-butadiene-styrene (ABS).

16. The breathing device of claim 15, wherein said breathing comprising a material selected from the group consisting of an acrylic, a polycarbonate, a polyethylene, a polyethylene terephthalate, a polyvinyl chloride, and a polystyrene.

17. A breather device comprising:
a breathing aid having a pair of nostril inserts and a connecting hook;
wherein said connecting hook connects said pair of nostril inserts;
wherein one of said pair of nostril inserts insertable into a nostril;
wherein another one of said pair of nostril inserts insertable into another nostril for opening nasal passages of a wearer;
wherein said breathing comprising a material selected from the group consisting of a high-density polyethylene (HDPE), a nylon, a heat-sealable polymer, a polypropylene and an acrylonitrile-butadiene-styrene (ABS); and
further wherein said connecting hook is flexible for expanding and contracting a distance between said pair of nostrils of said breathing aid.

18. The breather device of claim 17, wherein each one of said pair of nostril inserts having a circular shape.

19. The breather device of claim 18, wherein each one of said pair of nostril inserts comprise a grill structure.

20. The breather device of claim 19, wherein said connecting hook having a U-shape, wherein said connecting hook having a grip at a mid-section of said connecting hook for grasping by the wearer, and further wherein said mid-section of said connecting hook is thicker than end portions of said connecting hook.
